(19) United States
(12) Patent Application Publication
(10) Pub. No.: US 2024/0307490 A1
(43) Pub. Date: Sep. 19, 2024

(54) USE OF IL1-R1 DERIVED INHIBITOR OF IL-1B FOR THE TREATMENT OF IDIOPATHIC RECURRENT PERICARDITIS

(71) Applicant: R-Pharm Overseas, Inc., La Jolla, CA (US)

(72) Inventors: Mikhail Samsonov, Moscow (RU); Alexey Maslyansky, Saint Petersburg (RU); Sergey Grishin, Moscow (RU); Alexey Repik, Moscow (RU); Yan Lavrovsky, Del Mar, CA (US)

(21) Appl. No.: 18/672,826

(22) Filed: May 23, 2024

Related U.S. Application Data
(60) Provisional application No. 63/468,470, filed on May 23, 2023.
(60) Provisional application No. 63/345,142, filed on May 24, 2022.

(51) Int. Cl. A61K 38/17; A61K 9/00; A61P 9/00; A61P 29/00; A61P 37/06

(52) U.S. Cl. CPC A61K 38/1793; A61K 9/0019; A61P 9/00; A61P 29/00; A61P 37/06

(57) ABSTRACT

Disclosed invention provides for methods of treating idiopathic recurrent pericarditis (IRP), including a step of administering to a subject in need of treatment a heterodimeric IL-1R1/IL-1RAcP-derived composition at a therapeutically effective dose and an administration interval for a treatment period sufficient to reduce the risk of pericarditis recurrence relative to a control.

### BACKGROUND

Idiopathic recurrent pericarditis (IRP) is a rare autoinflammatory disease (see, for example, Blank N, Lorenz H M. Current Rheumatology Reports (2018)21:18; and https://www.orpha.net as assessed on Jan. 14, 2022) that has similar pathogenesis with adult-onset Still's disease and monogenic autoinflammatory diseases (see, for example, Imazio M. Heart. 2011 November, 97(22)1882-92; and Calabuig I J, Sanches Soriano R M, Marco Domingo T F et al. Rev Esp Cardiol. 2017, 70(3):208-219).

IL-1α and IL-1β are the pivotal cytokines in the pathophysiology of acute pericarditis and its recurrence (see Imazio M, Lazaros G, Gattorno M. European Heart Journal 2021, Epub ahead of print. PMID: 34528670). The teachings herein describe an analysis of clinical trial with Goflikicept-a heterodimeric IL-1R1/IL-1RAcP-derived composition, which binds both, IL-1α and IL-1β, in patients with IRP (see US Patent Pub. No. US20210403534A1, published on Dec. 30, 2021, the entire teachings of which are incorporated herein by reference).

### DETAILED DESCRIPTION

To evaluate the efficacy and safety of IL-1R1/IL-1RAcP-derived composition, treatment in patients with IRP, subjects with recurrence (n=9) or in inter-recurrence period (n=13) were enrolled in the run-in period of 12 weeks for treatment with NSAIDs and/or colchicine or 24 weeks for treatment with corticosteroids (CS). Dose finding approach was applied, where subjects from the first cohort (n=10) received subcutaneous IL-1R1/IL-1RAcP-derived composition, 80 mg every 2 weeks, subjects from the second cohort (n=12)-240 mg load within the first week, 80 mg within the second week, thereafter 80 mg every 2 weeks on top of background NSAIDs, colchicine and/or CS. Treatment response was considered as an achievement or maintenance of the following criteria: chest pain NRS≤3, CRP≤5 mg/L, absent or mild (<10 mm) pericardial effusion on Day 14. In responders NSAIDs/colchicine were stopped immediately, CS therapy was reduced and terminated within 12 weeks and for the next 12 weeks patients were on IL-1R1/IL-1RAcP-derived composition monotherapy. At the end of the run-in responders proceeded into the double-blind, placebo-controlled randomized-withdrawal (RW) period, and received 80 mg of IL-1R1/IL-1RAcP-derived composition every 2 weeks, or placebo. Primary endpoint was the time to the first pericarditis recurrence. In case of recurrence, the patients were unblinded, patients from placebo group retreated with IL-1R1/IL-1RAcP-derived composition. According to the analysis plan, efficacy/safety analysis included only unblinded data during the RW period

### Results

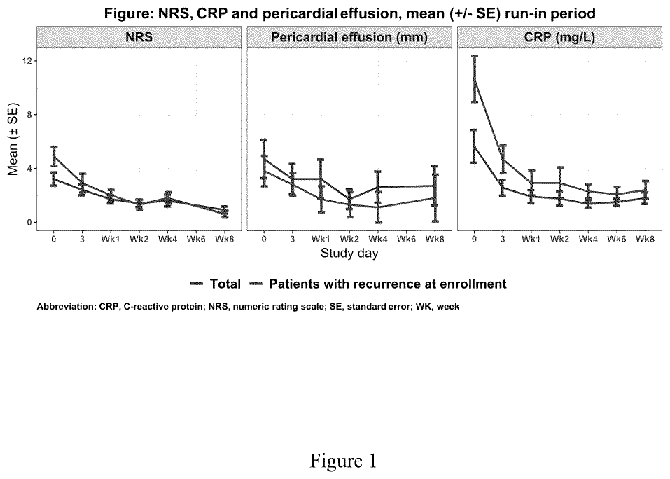

Treatment response was achieved in 8 out of 9 patients enrolled with recurrence, and all patients enrolled without recurrence remained in remission at Day 4. There were no new recurrence events on IL-1R1/IL-1RAcP-derived composition therapy, despite NSAIDs/colchicine/CS discontinuation during the run-in period. In the run-in period mean NRS, CRP and pericardial effusion decreased from baseline (see FIG. 1). Decrease of concentrations of IL-1RA, IL-6, calprotectin was reported during the run-in period, which confirms anti-inflammatory effect of IL-1R1/IL-1RAcP-derived composition. A total of 20 patients were randomized. Recurrent pericarditis occurred in 8 out of 10 patients in placebo group, there were no recurrence events in IL-1R1/IL-1RAcP-derived composition group up to the time of the analysis. After retreatment with IL-1R1/IL-1RAcP-derived composition and recurrence resolution there were no new recurrence events. Adverse events (AEs) occurred in 15 of 22 patients (68.2%) during the run-in period. During the run-in period, 6 out of 22 patients (27%) experienced infections, 4 (18.2%) cholesterol elevation, 3 (13.6%) lymphopenia, 2 (9.1%) lipase elevation, 2 (9.1%) injection site reactions. AEs occurred in 4 out of 8 patients (50%) during placebo treatment in the RW period. AEs occurred in 6 out of 8 patients (75%) after retreatment with IL-1R1/IL-1RAcP-derived composition. There were no deaths and no new safety signals. Overall safety profile was similar to those described for other IL-1 blockers.

The data show ability to achieve and maintain IRP remission on IL-1R1/IL-1RAcP-derived composition monotherapy with a favorable risk-benefit ratio.

What is claimed is:

1. A method of treating recurrent pericarditis comprising administering subcutaneously to a patient in need of treatment, a loading dose of a heterodimeric IL-1R1/IL-1RAcP-derived composition at 240 mg, followed by a maintenance dose of the heterodimeric IL-1R1/IL-1RAcP-derived composition at 80 mg once every two weeks, wherein the heterodimeric IL-1R1/IL-1RAcP-derived composition is goflikicept.
